(19) United States
(12) Patent Application Publication
(10) Pub. No.: US 2024/0318536 A1
(43) Pub. Date: Sep. 26, 2024

(54) MUD-GAS ANALYSIS FOR MATURE RESERVOIRS

(71) Applicant: Equinor Energy AS, Stavanger (NO)

(72) Inventors: Tao YANG, Stavanger (NO); Knut ULEBERG, Sandnes (NO); Nan CHENG, Trondheim (NO); Gulnar YERKINKYZY, Sandvika (NO)

(73) Assignee: Equinor Energy AS, Stavanger (NO)

(21) Appl. No.: 18/575,455

(22) PCT Filed: Jun. 29, 2022
(86) PCT No.: PCT/NO2022/050155

§ 371 Date: Dec. 29, 2023

(30) Foreign Application Priority Data
Jun. 30, 2021 (GB) 2109466.9

(51) Int. Cl. E21B 44/00

(52) U.S. Cl. CPC E21B 44/00; E21B 2200/22

(57) ABSTRACT

A method of generating a model for predicting at least one property of a fluid at a sample location within a hydrocarbon reservoir includes simulating behaviour of one or more hydrocarbon reservoirs during production; generating a plurality of simulated fluid samples from the one or more simulated hydrocarbon reservoirs, the plurality of simulated fluid samples corresponding to a plurality of different spatial locations and/or different time locations within the one or more simulated hydrocarbon reservoirs; generating a training data set including input data and target data based on the simulated fluid samples, the input data including simulated mud-gas data for each sample location indicative of mobile and immobile hydrocarbons at the sample location, and the target data including the at least one property of only the mobile hydrocarbons at each sample locations; and constructing a model using the training data set such that the model can be used to predict the at least one property of the fluid at a sample location based on measured mud-gas data for the sample location.

The present disclosure relates to the analysis of the hydrocarbon composition of reservoirs, and particularly to the analysis of the hydrocarbon composition of mature reservoirs based on mud-gas extracted when drilling through the reservoir.

Today, about two-thirds of the world's oil production comes from mature fields. Whilst the term “mature field” has no single definition, it is commonly understood to refer to fields in which production has reached its peak and has now started to decline. Sometimes, a “mature field” is defined as one in which the cumulative production has exceeded 50% of the initial 2P (proved plus probable) resources.

Although a good understanding of the initial reservoir fluid distribution is obtained from early discovery and appraisal wells, the remaining oil distribution in a mature field is complicated after many years of production with measures like pressure depletion, and gas and water injection. The remaining oils are often segmented and comparatively expensive to recover. The key to production success in a mature field is to accurately identify oil targets that can be recovered using cheap wells.

Often, horizontal wells are used to extract oil from mature reservoirs, as they can extract oil from a large area at comparatively low cost. Such wells can extend long distances horizontally, sometimes up to 10 km, and will pass through regions of the reservoir containing gases, such as gaseous hydrocarbons or injection gas, as well as regions of the reservoir containing liquid hydrocarbons.

It is undesirable to produce large quantities of free gas from an oil reservoir because the unwanted production gas will typically need to be compressed and re-injected into the reservoir, which adds significant cost to the operation and leads to significant CO₂ emission. In order to avoid producing free gas from the reservoir, the casing of a horizontal production well is ideally perforated only at locations within oil regions. If a gas-containing region is perforated, then the production gas-oil ratio of the well will be very high due to the high mobility of the gas phase.

Whilst many techniques exist for examining the composition of new reservoirs during the exploration stage, the available techniques for reservoir composition analysis within mature fields during production phase is much more limited.

Often, 4D seismic analysis is used to identify reservoir fluid types within mature fields. This is a form of time-lapse seismic analysis that comprises capturing 3D seismic survey data from the field at time-spaced intervals, often 6-month intervals, and examining changes in the data with time.

The use of multiple, time-spaced data sets allows for a 3D model of the fluid distribution within the reservoir to be produced by updating the initial reservoir fluid distribution model to account for changes over time. However, 4D seismic interpretation does not provide quantitative reservoir fluid properties data, but rather a qualitative indication of fluid changes, caused by any one or more of pressure changes, density changes and saturation changes. Many assumptions must be made to interpret what these changes mean (e.g. gas displacing oil, or water displacing oil), and so there is a high degree of uncertainty associated with these models.

Petrophysical logs are used extensively to identify reservoir fluid types. Density-neutron separation data presented in petrophysical logs can be utilized to distinguish oil and gas. However, density-neutron logs are responsive to both lithology and reservoir fluids and therefore, there are uncertainties related to the interpretation from petrophysical logs based on such data. Regarding the mature reservoirs, the method becomes very challenging due to co-existing oil and gas phases at a specific well depth.

Techniques such as sampling while drilling and downhole fluid sampling are not well suited to the horizontal wells used in mature fields, due to the length of the wells and the fact that the wells are not oriented vertically.

A new technique has been proposed in WO 2020/185094 A1, whereby a machine-learning model is used to predict one or more properties of the reservoir fluid, such as the gas-oil ratio or the average density, based on measured mud-gas data acquired whilst drilling exploration wells through a new reservoir.

Mud-gas logging is a technique in which hydrocarbon gas is released from drilling mud at the surface and then examined. When drilling into the reservoir, a small quantity of the reservoir fluid will be carried in the drilling mud to the surface. At the surface, the drilling mud is processed to release a mixture of gases, known as “mud gas”, which is then examined to estimate certain properties of the reservoir.

The technique proposed in WO 2020/185094 A1 has been found to provide a high degree of accuracy for reservoir fluids in their initial state. However, attempts to apply this technique to mature fields has found the predictions to be much less reliable.

Consequently, a need exists for a new technique that can determine properties of reservoir fluid within mature reservoir in order to identify regions containing liquid hydrocarbons suitable for production.

Viewed from a first aspect, the present invention provides a method of generating a model for predicting at least one property of a fluid at a sample location within a hydrocarbon reservoir, the method comprising: simulating behaviour of one or more hydrocarbon reservoir during production; generating a plurality of simulated fluid samples from the one or more simulated hydrocarbon reservoir, the plurality of simulated fluid samples corresponding to a plurality of different spatial locations and/or different time locations within the one or more simulated hydrocarbon reservoir; generating a training data set comprising input data and target data based on the simulated fluid samples, the input data comprising simulated mud-gas data for each sample location indicative of mobile and less mobile hydrocarbons at the sample location, and the target data comprising the at least one property of only the mobile hydrocarbons at each sample locations; and constructing a model using the training data set such that the model can be used to predict the at least one property of the fluid at a sample location based on measured mud-gas data for the sample location.

The hydrocarbon reservoir may be a mature reservoir. The hydrocarbon reservoir may have undergone production for six or more months, optionally two or more years, and further optionally five or more years. The cumulative production of the hydrocarbon field may have exceeded 50% of the initial combined proven and probable oil reserves within the hydrocarbon field. The hydrocarbon field may have undergone gas injection. The hydrocarbon reservoir may comprise at least one gas-flooded reservoir.

This method recognises that reservoir fluid, particularly within mature reservoirs, may comprise a mixture of relatively mobile fluids and relatively less mobile fluids.

Mud-gas data is collected when drilling through the reservoir fluid and is therefore indicative of the overall composition of the reservoir fluid, including both the mobile fluids and the immobile fluids. However, when producing from the reservoir, the mobile fluids will typically form the bulk of the production fluid, with the relatively less mobile fluids remaining substantially stationary. Hence, it is desirable to identify specific properties of just the mobile fluids based on the mud-gas data.

It is difficult to obtain accurate training data that discriminates between the mobile and immobile fluids based on actual measured data samples. However, the inventors have identified that by generating simulated training data, it is possible to generate a model that accurately determines properties of the mobile fluid based on mud-gas data.

The simulated fluid samples represent the composition of the reservoir fluid at the respective sample location within the simulated reservoir. The simulated fluid samples may comprise multiphase fluid i.e. both oil and gas. The sample may thus comprise a single-phase mobile fluid of one phase and a single-phase immobile fluid of a different phase and the composition of the total free fluid composition will correspond to the gas-oil ratio of the mobile phase, or, the sample may comprise a mobile oil phase and a mobile gas phase and the composition of the total free fluid composition will correspond to the gas-oil ratio of the gas phase.

Simulated mud-gas data may comprise the C₁ to C₅ concentrations of both the mobile and immobile fluid present in the simulated fluid samples. The simulated mud-gas data therefore simulates fully corrected mud-gas data from an advanced mud-gas logging operation. Fully corrected mud-gas data is raw mud-gas data adjusted by a recycling correction and an extraction efficiency correction. Measured mud-gas data comprises data relating to the C₁ to C₅ concentrations of hydrocarbon gas released from drilling mud following its use within a wellbore at a drilling site. The measured mud-gas data is preferably advanced mud-gas data, also known as fully corrected mud-gas data, but is some embodiments the method may use standard mud-gas data, sometimes known as raw mud-gas data.

The input data may comprise the combined C₁ to C₅ concentrations of both the mobile and immobile phase of the simulated fluid sample, and the target data may comprise a property, for example a gas-oil ratio, of the mobile fluid at the sample location.

In accordance with this method, a property of a mobile fluid within an oil reservoir can be predicted from mud-gas data using a machine learning model. The predicted fluid property can be used in order to identify regions of the reservoir containing liquid hydrocarbons suitable for production.

In some embodiments, the plurality of simulated fluid samples correspond to time locations comprising one or more of: a time when the simulated reservoir is at an initial state; a time when the simulated reservoir is undergoing or has undergone pressure depletion; a time when the reservoir is undergoing or has undergone water injection; and a time when the reservoir is undergoing or has undergone gas injection. Optionally, the time locations may include multiple time locations within one or more of the production processes.

A reservoir in an initial state has not yet undergone any production processes.

Pressure-depletion is a process by which reservoir fluid can be produced from a reservoir, the reservoir fluid is driven under the natural pressure of the reservoir to flow towards a production well in order to be extracted.

Water injection is a process by which reservoir fluid can be produced from a reservoir. Injection fluid comprising water is introduced into the reservoir via an injection well, the pressure and flow of the injection fluid encourages reservoir fluid to flow towards the production well where it is extracted.

Gas injection is a process by which reservoir fluid can be produced from a reservoir. Injection fluid comprising gas is introduced into the reservoir via an injection well, the pressure and flow of the injection fluid encourages reservoir fluid to flow towards the production well where it is extracted.

The simulated fluid samples can therefore be representative of the reservoir at various stages of the lifecycle of the reservoir. By generating simulated fluid samples multiple time locations of the reservoir over time, a wide range of reservoir conditions can be examined when training the model. In particular, the effects of the production processes, including pressure depletion, water injection and gas injection, on the composition of the reservoir can be simulated in order to allow use of the model in reservoirs within a reservoirs having undergone these processes.

In some embodiments, the at least one property comprises a gas-oil ratio of the fluid at the sample location, and preferably a gas-oil ratio of the mobile fluid at the sample location.

It will be understood that a gas-oil ratio refers to a ratio between the quantity of gaseous hydrocarbon and the quantity of liquid hydrocarbon at surface conditions. The gas-oil ratio is preferably a volume ratio.

Thus, the model can be used to predict the gas-oil ratio of the mobile fluid present within the reservoir at a given sample location based on the measured mud-gas data which corresponds to that sample location. Producing free gas from an oil reservoir is generally to be avoided, therefore predicting the gas-oil ratio of the mobile fluid within the reservoir is important so that producing from locations in the reservoir that would produce gas, i.e. locations comprising mobile fluid that has a high gas-oil ratio, can be avoided and producing from locations that would produce oil, i.e. locations comprising mobile fluid that has a low gas-oil ratio, can be exploited.

In some embodiments, the at least one property comprises a density of the fluid at the sample location, and preferably a density of the mobile fluid at the sample location.

In some embodiments, the at least one property comprises a saturation pressure of the fluid at the sample location, and preferably the saturation pressure of the mobile fluid at the sample location.

In some embodiments, the at least one property comprises a formation volume factor of the fluid at the sample location, and preferably a formation volume factor of the mobile fluid at the sample location.

It will be understood that the formation volume factor is the ratio of the volume of gas present at reservoir conditions, i.e. the conditions at the sample location such as pressure and temperature, to the volume of gas present at standard conditions, i.e. the conditions at the surface of the well following production.

In some embodiments, the at least one property comprises a concentration of a C₇₊ hydrocarbon within the fluid at the sample location, and preferably a concentration of a C₇₊ hydrocarbon within the mobile fluid at the sample location. Optionally, the at least one property may comprise concentrations of multiple C₇₊ hydrocarbons within the fluid at the sample location, and preferably may comprise concentrations of multiple C₇₊ hydrocarbons within the mobile fluid at the sample location.

In some embodiments, the mud-gas data is indicative of a concentration of C₁ to C₅ hydrocarbon gases at the sample location. Preferably the mud-gas data comprises data relating to the concentration of C₁, C₂, C₃, iC₄, nC₄, iC₅, and nC₅ hydrocarbon gases at the sample location. The mud-gas data may hence comprise the concentration of at least one of methane, ethane, propane, iso-butane, normal butane, iso-pentane and normal pentane.

In some embodiments, simulating the behaviour of one or more hydrocarbon reservoir during production is performed using slim-tube simulations.

The slim-tube simulations may produce compositional data representative of the reservoir fluid at a given location. Slim-tube simulations may involve modelling a section of the reservoir as a tube, such as a cylinder, filled with a simulated porous media, the pores of which are saturated with a simulated reservoir fluid. The simulation may further involve simulating the introduction of a fluid representing injection fluid into the tube and simulating how the injection fluid and reservoir fluid interact, and in particular how the reservoir fluid is displaced by the injection fluid. The simulation within the slim tube can therefore be considered as representative of a flow path of the reservoir fluid between an injection well and a production well in one of the one or more simulated hydrocarbon reservoirs.

The simulation may use an equation of state model, and preferably a tuned equation of state model, which may be tuned to a specific oil field.

The simulating of the behaviour of one or more hydrocarbon reservoir during production may comprise running a plurality of slim-tube simulations, for example at least 1,000 slim-tube simulations, and optionally at least 10,000 slim-tube simulations. The slim-tube simulations may use a plurality of simulated reservoir fluids representative of reservoir fluids known to exist within a specific oil field. The slim-tube simulations may use a plurality of simulated porous media representative of porous media known to exist within a specific oil field.

The slim-tube may be separated into grid cells and the composition of the fluid corresponding to each grid cell is monitored over time as the injection fluid travels through the slim tube.

Viewed from a second aspect, the present invention provides a computer-based model for predicting at least one property of a fluid at a sample location within a hydrocarbon reservoir based on measured mud-gas data for that sample location, the computer-based model having been generated by a method as described above.

Viewed from a third aspect, the present invention provides a tangible computer-readable medium storing the computer-based model.

Viewed from a fourth aspect, the present invention provides a method of predicting a value of a fluid property of a fluid at a sample location within a hydrocarbon reservoir, the method comprising: providing measured mud-gas data for the sample location; and predicting the values of a fluid property of the fluid at the sample location by supplying the measured mud-gas data to the model.

Preferably the predicted value of a fluid property relates to the mobile fluid present at the sample location. The model being the computer-based model described above.

Viewed from a fifth aspect, the present invention provides a method of predicting a value of a fluid property of a fluid along a length of a well through a hydrocarbon reservoir, the method comprising: predicting a value of a fluid property of a fluid at a plurality of sample locations along a length of a well using the method described above. Preferably the predicted value of a fluid property relates to the mobile fluid present at each respective sample location.

In some embodiments, the method further comprises displaying, using an electronic display screen, a graph plotting the predicted value of the fluid property against a location of the respective sample location for each of the plurality of sample locations along the length of the well.

Viewed from a sixth aspect, the present invention provides a method of generating a model for predicting at least one property of a fluid at a sample location within a hydrocarbon reservoir, comprising: simulating behaviour of one or more hydrocarbon reservoir during production; generating a plurality of simulated fluid samples from the one or more simulated hydrocarbon reservoir, the plurality of simulated fluid samples corresponding to a plurality of different spatial locations and/or different time locations within the one or more simulated hydrocarbon reservoir; generating a training data set comprising input data and target data based on the simulated fluid samples, the input data comprising simulated mud-gas data for each sample location indicative of mobile and less mobile hydrocarbons at the sample location, and the target data comprising the at least one property of only the mobile hydrocarbons at each sample locations; and correlating the input data against the output data to construct a model using the training data set such that the model can be used to predict the at least one property of the fluid at a sample location based on measured mud-gas data for the sample location.

Preferably the predicted value of a fluid property relates to the mobile fluid present at the sample location.

Viewed from a seventh aspect, the present invention provides a method comprising: drilling a well bore through a hydrocarbon reservoir, wherein mud-gas data is collected as the well is drilled; predicting a value of a fluid property of a fluid at a plurality of sample locations along a length of the well bore using a method as described above; determining at least one perforation location along the well bore, based on the predicted value of the fluid property of the fluid; and perforating a casing of the well bore at the determined at least one perforation location.

Preferably the predicted value of a fluid property relates to the mobile fluid present at the sample location.

The method therefore comprises determining one or more perforation locations within a well bore based on the predicted value of a fluid property predicted using measured mud-gas data. That is to say, one or more locations where a casing of the well bore is perforated to permit inflow of reservoir fluid. By using the method described above, the property of the mobile fluid at a specific location within the reservoir can be predicted much more accurately, thereby allowing precise perforation of the well bore in regions comprising oil, whist avoiding perforation of the well bore in regions comprising free gas. For example, the determining the one or more perforation locations may comprise determining that a gas-oil ratio at the location is below a predetermined threshold value based on the predicted fluid property.

In some embodiments, the well bore is a horizontal well bore.

A horizontal well bore may comprise at least one section oriented at an angle greater than 80° with respect to vertical. Horizontal well bores can be particularly important for mature wells in which the remaining oil reserves may become difficult to access using vertical wells. The perforation locations may be located in a horizontal section of the well bore.

In some embodiments, the well bore is a production well bore.

A production well bore is used to extract reservoir fluid from the reservoir, and transport the fluid to the surface. When the reservoir is undergoing production processes such as water and/or gas injection, a production well bore may be used in conjunction with an injection well bore. The injection well bore is used to introduce injection fluid (for example water or gas) in to the reservoir and the injection fluid encourages the reservoir fluid towards the production well bores to be extracted.

In some embodiments, the reservoir is a mature reservoir.

In some embodiments the hydrocarbon field has undergone production for six or more months, optionally two or more years, and further optionally five or more years. The cumulative production of the hydrocarbon field may have exceeded 50% of the initial combined proven and probable oil reserves within the hydrocarbon field. The hydrocarbon reservoir may have undergone gas injection, and may comprise at least one gas-flooded reservoir.

Certain preferred embodiments of the invention will now be described in greater detail, by way of example only and with reference to the accompanying drawings, in which:

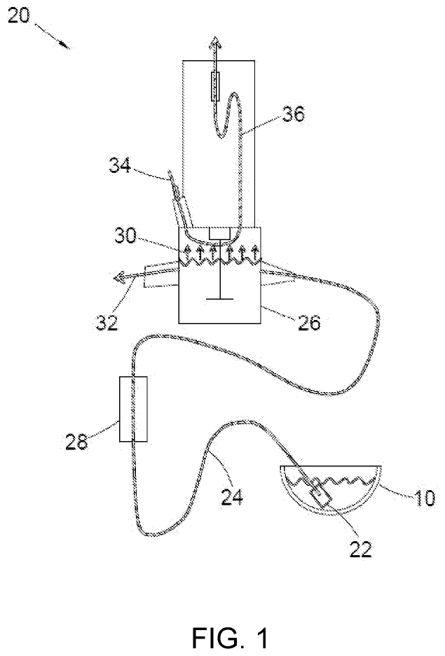

FIG. 1 is a schematic illustration of a mud-gas analysis tool; and

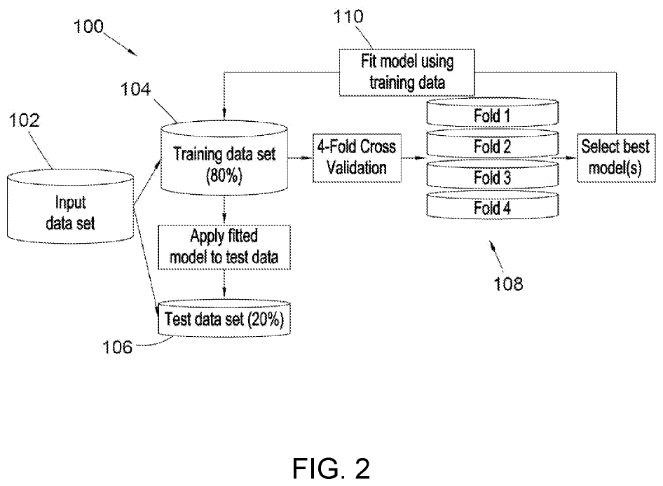

FIG. 2 illustrates a workflow for a machine learning algorithm to generate a first model for predicting a gas oil ratio using a training data set.

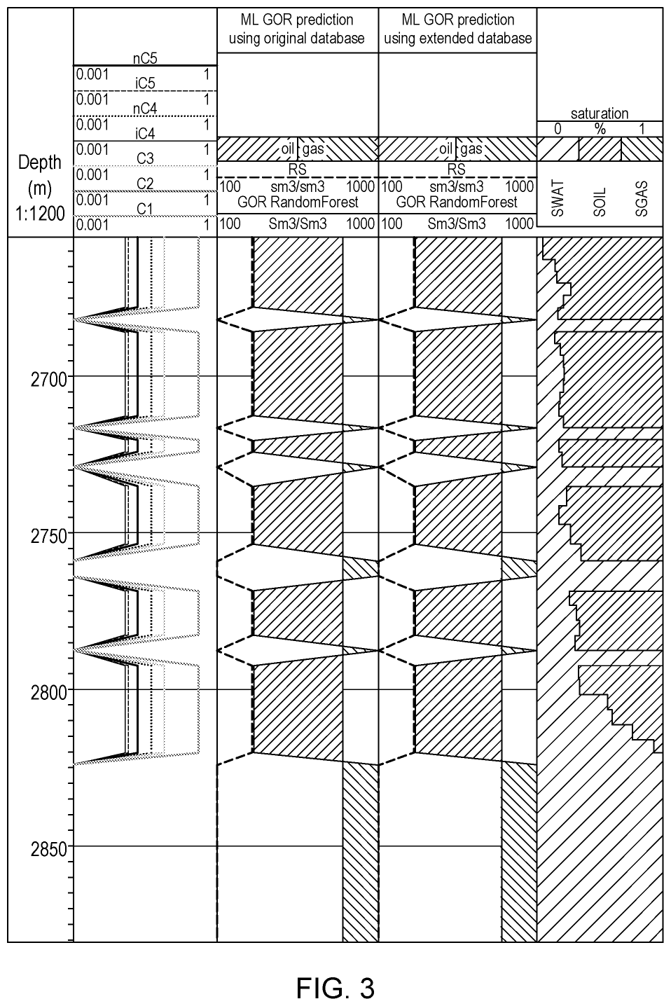

FIG. 3 shows a comparative chart of the gas oil ratio of free fluid at a location within a reservoir predicted using two different models, for a reservoir at an initial reservoir state.

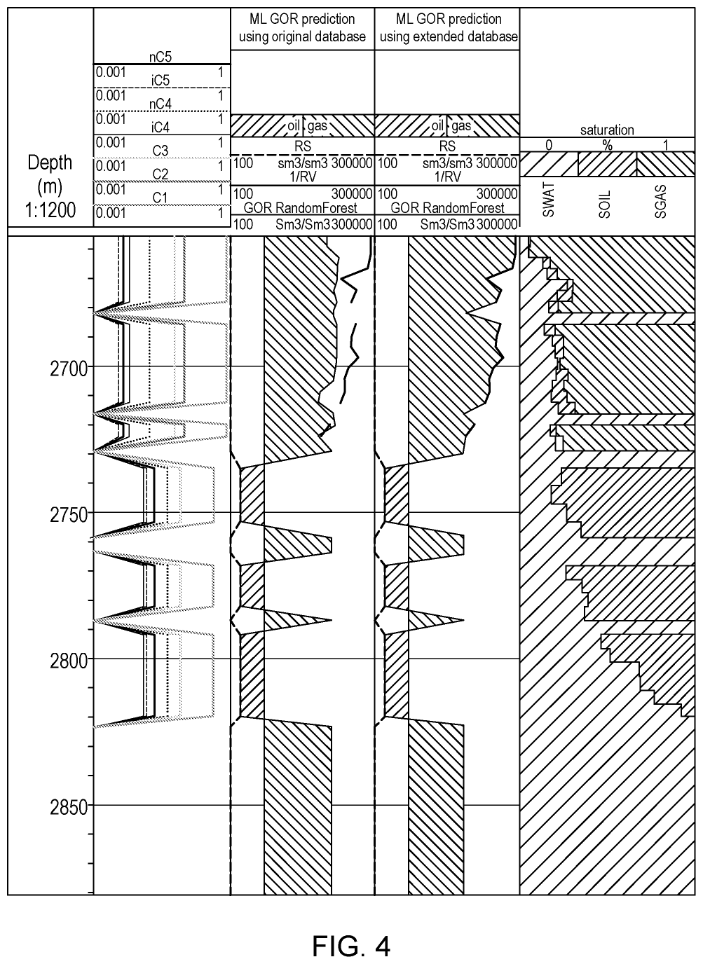

FIG. 4 shows a comparative chart of the gas oil ratio of free fluid at a location within a reservoir predicted using two different models, for a reservoir at a time following a period of gas injection.

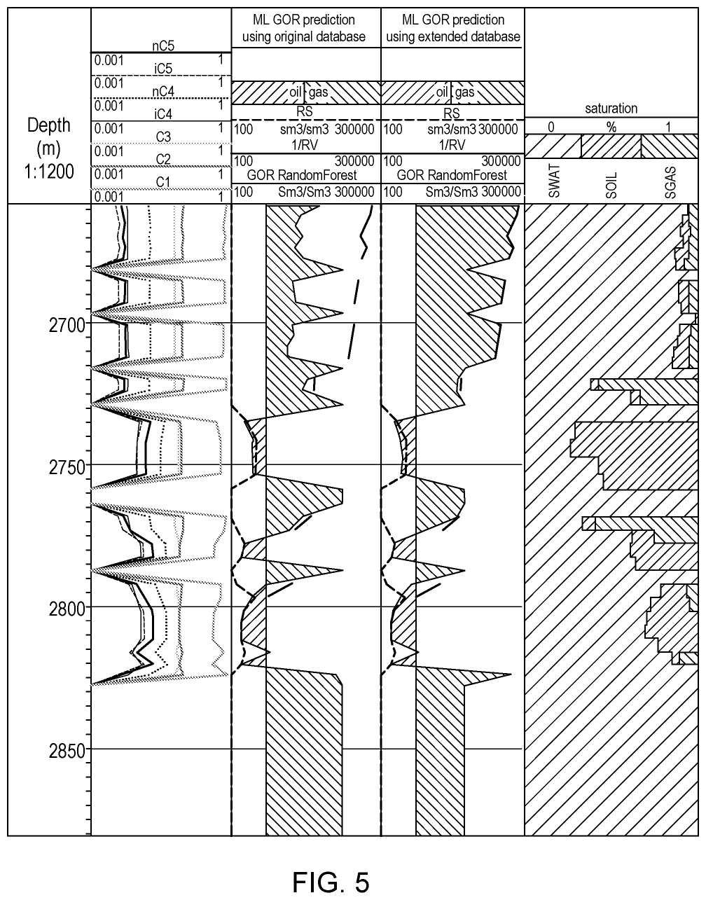

FIG. 5 shows a comparative chart of the gas oil ratio of free fluid at a location within a reservoir predicted using two different models, for a reservoir at a time following a period of gas and water injection.

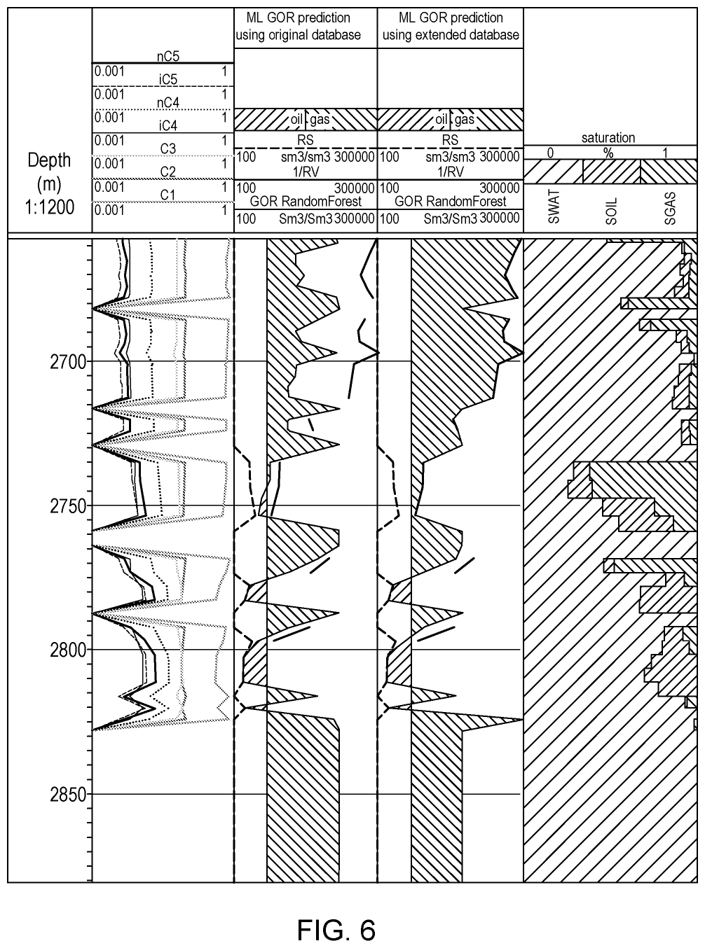

FIG. 6 shows a comparative chart of the gas oil ratio of free fluid at a location within a reservoir predicted using two different models, for a reservoir at a time where there is a mobile oil and a mobile gas phase.

Drilling fluid is a fluid used to aid the drilling of boreholes into the earth. The main functions of drilling fluid include providing hydrostatic pressure to prevent formation fluids from entering into the well bore, keeping the drill bit cool and clean during drilling, carrying out drill cuttings, and suspending the drill cuttings while drilling is paused and when the drilling assembly is brought in and out of the hole.

Drilling fluids are broadly categorised into water-based drilling fluid, non-aqueous drilling fluid, often referred to as oil-based drilling fluid, and gaseous drilling fluid. The present disclosure is particularly applicable to liquid drilling fluid, i.e. water-based drilling fluid or non-aqueous drilling fluid, which is commonly referred to as “drilling mud”.

Mud-gas logging entails gathering data from hydrocarbon gas detectors that record the levels of gases brought up to the surface in the drilling mud during a bore drilling operation.

Conventional mud-gas logging is used to identify the location of oil and gas zones as they are penetrated, which can be identified by the presence of gas in the mud system. This may be used to provide a general indication of the type of reservoir, as well as to determine where to take downhole fluid samples for more detailed analysis of the fluid composition.

The presence of hydrocarbon gas may be detected, for example, with a total gas detector. Once the presence of hydrocarbon gas is detected, its composition may be examined for example with a gas chromatograph. The detection of the composition of the mud gas described below is sometimes referred to as “advanced mud-gas logging”.

The most common gas component present is usually methane (C₁). The presence of heavier hydrocarbons, such as C₂ (ethane), C₃ (propane), C₄ (butane) and C₅ (pentane) may indicate an oil or a “wet” gas zone. Even heavier molecules, up to about C₇ (heptane) or C₈ (octane), may also be detectable, but are typically present only in very low concentrations. Consequently, the concentrations of these hydrocarbons are often not recorded.

The composition of the mud gas can be examined in order to provide predictions of the C₁ to C₅ concentrations within the reservoir fluid.

The measured mud-gas data is usually referred to as “raw” mud-gas data and is not comparable to the actual composition of the reservoir, since the mud gas contains gases that do not originate from the reservoir (e.g. gases present in the drilling mud or remaining from previous injection when recycling the drilling mud) and also because lighter hydrocarbon (e.g. C₁) are carried more easily by the drilling mud than heavier hydrocarbons (e.g. C₂ to C₅).

Firstly, a recycling correction is made to eliminate contamination by gases originating from previous injections of the drilling mud. This correction is applied based on a separate mud-gas measurement that was taken before the drilling mud was injected into the drilling string.

Secondly, an extraction efficiency correction step is applied to increase the concentration of intermediate components (from C₂ to C₅), such that the mud-gas data after this step closely resembles a corresponding reservoir fluid sample composition.

The mud-gas-data after hydrocarbon recycling correction and extraction efficiency correction is usually referred to as “fully corrected” mud-gas data.

It will be appreciated that there is a lag-time between the drill bit passing through the sample location, and when the mud reaches the surface and is analysed. However, workers in this field will be familiar with the procedures for calculating the lag time to determine the depth to which the mud-gas sample corresponds. Therefore, this will not be discussed in detail.

An exemplary mud-gas analysis tool 20 is shown schematically in FIG. 1.

The tool 20 is coupled to a flow line 10 containing drilling mud returned from a borehole of a well. As discussed above, the drilling mud may be water-based mud or oil-based mud.

The tool 20 comprises a sampling probe 22 disposed with respect to the flow line 10 so as to collect a sample 24 of the drilling mud from the flow line 10. The drilling mud sample 24 is preferably a continuous sample, i.e. such that a portion of the flow of drilling mud within the flow line 10 is diverted through the mud-gas analysis tool 20.

The drilling mud sample 24 is supplied to a gas-separation chamber 26 where at least a portion of the gas carried by the drilling mud is released. The sample of drilling mud may be heated by a heater 28 upstream of the gas-separation chamber 26. Heating the drilling mud sample 24 helps to release the gas from the drilling mud sample 24. Typically, the mud sample 24 is heated to a temperature of around 80° C. to 90° C.

The released gas 30 is directed from the separation chamber 26 to a gas analysis unit (not shown), while the degassed mud 32 is returned to the flow line 10 or to another location for re-use.

The gas analyser may comprise a total gas detector, which may provide a basic quantitative indication as to how much gas is being extracted from the drilling mud by the tool 20. Total gas detection typically incorporates either a catalytic filament detector, also called a hotwire detector, or a hydrogen flame ionization detector.

A catalytic filament detector operates on the principle of catalytic combustion of hydrocarbons in the presence of a heated platinum wire at gas concentration below the lower explosive limit. The increasing heat due to combustion causes a corresponding increase in the resistance of the platinum wire filament. This resistance increase may be measured through the use of a Wheatstone bridge or equivalent detection circuit.

A hydrogen flame ionization detector functions on the principle of hydrocarbon molecule ionization in the presence of a very hot hydrogen flame. These ions are subjected to a strong electrical field resulting in a measurable current flow.

The gas analysis device may additionally or alternatively comprise an apparatus for detailed analysis of the hydrocarbon mixture. This analysis is usually performed by a gas chromatograph. However, several other detecting devices may also be utilised including a mass spectrometer, an infrared analyser or a thermal conductivity analyser.

A gas chromatograph is a rapid sampling, batch processing instrument that provides a proportional analysis of a series of hydrocarbons. Gas chromatographs can be configured to separate almost any suite of gases, but typically oilfield chromatographs are designed to separate the paraffin series of hydrocarbons from methane (C₁) through pentane (C₅) at room temperature, using air as a carrier. The chromatograph will report (in units or in mole percent) the quantity of each component of the gas detected.

A carrier gas stream 34, commonly comprising air, may be supplied to the separation chamber 26 and mixed with the released gas 30 to form a gas mixture 36 that is supplied to the gas analysis unit. The carrier gas stream 34 provides a continuous flow of carrier gas in order to provide a substantially continuous flow rate of the gas mixture 36 from separation chamber 26 to the gas analysis unit. Additionally, in the case of a gas analyser comprising a combustor, the use of air as the carrier gas may provide the necessary oxygen for combustion.

In some arrangements, the tool 20 may be configured to detect and/or remove H₂S from the gas to prevent adverse effects that could influence hydrocarbon detection.

In some embodiments, non-combustible gases, such as helium, carbon dioxide and nitrogen, can be detected by the gas analyser in conjunction with the logging of hydrocarbons.

Mud-gas logging was commonly performed when drilling exploration wells in a newly identified reservoir in order to identify reservoir fluid type. This information could then be used to guide the selection of location for performing downhole fluid sampling.

Recent innovations by the applicant, as discussed in in WO 2020/185094 A1, have shown that it is possible to identify certain properties of the reservoir fluid from the mud-gas data, such as density and gas-oil ratio, with a high degree of precision under initial reservoir conditions.

Whilst mud-gas logging is less commonly used when drilling production wells, it is comparatively cheap to implement because it does not require interruption of the drilling process to analyse the reservoir fluid. It would therefore be desirable if mud-gas analysis techniques could be used within mature fields to identify the reservoir composition along the length of the well. However, when the above techniques were applied to production wells drilled in mature fields, it was found that the precision of the estimates was significantly reduced compared to when the analysis was applied in exploratory wells.

Following investigation as to why the mud-gas models showed less accuracy within mature fields, the inventors have identified that, within a mature field, there is often a co-existing of both gas and oil phases within the reservoir in the form of mobile fluid and immobile fluid.

The mobile fluid is fluid that can flow relatively freely within the reservoir, for example as the reservoir is undergoing pressure depletion or by the action of gas or water injection. This mobile fluid is the fluid that is produced from the reservoir during production, and it is the composition of this fluid that is of interest when examining the reservoir composition.

The immobile fluid is fluid that is trapped within the rock formation of the reservoir, and is therefore significantly less mobile than the mobile fluid. Hence when production is being carried out, the immobile fluid is not produced and will typically remain substantially stationary within the reservoir.

The term critical saturation refers to the minimum saturation of fluid within a porous media required for continuous flow of fluid through that media.

When a well is drilled through a reservoir, the drill bit breaks down the rock formation of the reservoir releasing the immobile fluid. Consequently, the mud-gas data collected is indicative of the combined composition of the mobile and immobile fluids.

Within a new reservoir, where the reservoir has had millions of years to reach an equilibrium state, the gas and liquid present in the reservoir partitions into separate phases such that, at any specific reservoir location, only a single phase of either gas or liquid is present. Consequently, the mobile and immobile fluids at each location within the reservoir have substantially the same composition corresponding to the single phase gas or oil present.

During production, only the mobile fluid will be displaced and the immobile fluid will remain at the same location. Therefore, within a mature reservoir, the compositions of the mobile and immobile fluids will deviate from one another.

The model for performing mud-gas analysis discussed above was generated using data collected at initial reservoir conditions. However, in a mature field, this model no longer applies. Therefore, it is necessary to generate a new model for analysis of the reservoir fluid composition within a mature field.

As discussed above, downhole fluid analysis is difficult to perform in a mature field. Furthermore, the wells in such fields will often produce fluid from multiple locations within the reservoir, mixing the fluids from each of these locations. Consequently, it is also not possible to accurately determine the reservoir fluid composition for a particular location within a reservoir from examination of the production fluid.

In order to obtain mud-gas data and mobile fluid composition data within a mature reservoir, a plurality of reservoirs were simulated over their lifecycle using compositional simulation modelling.

The simulated reservoirs were simulated from an initial state, through production under pressure depletion, production under water injection, and production under gas injection. It will be appreciated that optionally one or more of these production states may be omitted.

In the initial state, the mobile and immobile fluids at each location within the simulated reservoir have substantially the same composition corresponding to the single phase gas or oil present. During a reservoir's production lifecycle, water and/or gas injection may be used to stimulate the production of oil from the reservoir. The composition of the fluid within the simulated reservoir will therefore change as the injection fluid is introduced, and the compositions of the mobile and immobile phases will deviate from one another as the mobile fluid is displaced.

The injected fluid aids in the continued production of oil from the reservoir by increasing the depleted reservoir pressure, as well as by encouraging the oil to flow. Injection wells are drilled into the reservoir, and through these injection wells fluid is pumped into the reservoir. The injection fluid encourages oil in the reservoir towards the production wells where the oil is extracted.

Entire reservoirs can be simulated by implementing an equation of state model, and by using a compositional reservoir simulator to obtain simulated reservoir fluid properties data for the reservoir fluid as the reservoir undergoes production.

Reservoir fluid properties data represents the composition of a fluid sample from a reservoir, typically including the composition in terms of each of C₁ to C₃₆₊ hydrocarbons. Reservoir fluid properties data is sometimes referred to as PVT data because measured reservoir fluid properties data is commonly obtained in a pressure-volume-temperature (PVT) laboratory, where researchers will employ various instruments to determine reservoir fluid behaviour and properties from the reservoir samples.

An equation of state model defines the relationship between pressure, volume and temperature for the fluid within a reservoir and can be used to determine the phase of a particular fluid sample. An equation of state model will typically have anywhere from 10 to 30 components, corresponding to the fluid composition. For example, a 14-component equation-of-state model may comprise the following components: N₂, CO₂, C₁, C₂, C₃, iC₄, nC₄, iC₅, nC₅, C₆, C₇-C₉, C₁₀-C₁₅, C₁₆-C₂₉, and C₃₀₊. By supplying the composition of a particular reservoir fluid sample to the equations of state model, it is possible to predict how that fluid sample will behave under various conditions.

Typically, a tuned equations of state model is available for a mature reservoir. This is developed by gathering fluid samples from a large number of samples collected from the exploration and appraisal wells associated with the mature field. Equation of state parameters are then modified from default or initial estimations using a regression procedure to match lab-reported reservoir fluid properties measurements. The tuned equations of state model is tailored to the specific oil field.

Simulating an entire reservoir over its full production lifetime, in order to model the compositional changes in the reservoir fluid during each stage of production, is time consuming and computationally complex and expensive. This means that in order to produce a workable simulation in an acceptable time, the full equation of state comprising components representing each of the individual hydrocarbon components from C₁ to C₃₆₊ is not normally used. Instead, when modelling an entire reservoir, the components used in the equations of state are typically reduced to between 5 and 8.

Reservoir models based on the compressed equation of state may be suitable for large scale simulations. However, they do not produce data that is sufficiently accurate for the fine scale model required here. Specifically, compressing the components used in the calculation results in the grouping of the C₁ to C₅ components. Data relating to the C₁, C₂, C₃, iC₄, nC₄, iC₅, nC₅ components are then no longer distinguishable and hence the grouped data cannot be used for comparison with mud-gas data which contains these components. The equation of state model simplified in this way therefore cannot be used to generate date for training a machine learning model for prediction of a gas-oil ratio of free fluid at a location within a reservoir based on measured mud-gas data.

Instead, slim-tube simulations were used to generate simulated fluid samples representing the flow path of the reservoir fluid between injection wells and production wells. Slim-tube tests are computationally simple and can therefore utilise the full, tuned equations of state model to provide highly accurate estimations of the reservoir fluid properties data across the lifecycle of a simulated reservoir.

In a laboratory setting, physical slim-tube tests were carried out by filling a long coiled tube with a porous media, such as sand with a given mesh size, which may be varied to produce desired test conditions. The resulting open pores of the tube were then saturated with the desired reservoir oil and maintained at a given temperature and/or pressure which again may be varied to produce desired test conditions. The flow of the free fluid within the slim-tubes as the injection fluid is introduced allows the displacement of the reservoir fluid to be simulated.

Injection fluid of varying compositions, such as gas and/or water injection, is injected at the inlet of the slim-tube at a range of pressures. The slim-tube is separated into grid cells and the compositional data corresponding to each grid cell is monitored. The movement of the reservoir fluid and its interaction with the injection fluid can hence be tracked.

In the present method, compositional simulations of slim-tube tests were used to enable a sufficiently large data set to be obtained efficiently representing the change in the fluid composition as the reservoir undergoes production.

The simulated slim-tube tests were performed across a large range of test conditions (in the region of 100,000 tests) comprising different fluid compositions and reservoir conditions (for example the pressure, temperature and porous media). The fluid compositions and reservoir conditions were selected based on typical compositions and conditions found within the oil field being examined, and the simulations were performed using a tuned equation of state model for that oil field, as discussed above. Therefore, the simulations closely corresponded to the conditions arising in the specific oil field.

The fluid in a simulated slim-tube test is present as either a mobile fluid, which moves under the influence of the injection fluid, or, as an immobile fluid which remains within the pores of the porous media within the slim-tube. The mobile fluid corresponds to the fluid which would make up the majority of the production fluid extracted from the well during production, and the immobile fluid corresponds to fluid which is either not present in the production fluid or is present in the production fluid only as a small proportion.

Typically, the composition of the immobile fluid corresponds closely to the composition of the fluid at the initial conditions, and is therefore substantially single phase. However, the composition of the mobile fluid can vary substantially over time, and may sometimes comprise a multi-phase fluid.

Where the reservoir simulation indicates the presence of multi-phase fluid, there are three possible situations. In the case of a single-phase mobile fluid and a single-phase immobile fluid of a different phase, two situations arise:
a) For the case where the mobile fluid comprises oil and only residual gas is present as the immobile fluid, the composition of the total free fluid composition will be close to the oil phase composition. Therefore, the predicted gas-oil ratio of the free fluid will correspond to the gas-oil ratio of the oil.
b) For the case where the mobile fluid comprises gas and only residual oil is present as the immobile fluid, the composition of the total free fluid composition is close to the gas composition. Therefore, the predicted gas-oil ratio of the free fluid will correspond to the gas-oil ratio of the gas.

In a multi-phase scenario, the mobile fluid comprises a mobile oil phase and a mobile gas phase. When the mobile fluid is multi-phase, the following situation will arise:
c) For the case where both oil and gas are present at a significant saturation percentage, i.e. above a critical saturation percentage, the composition of the total free fluid composition will correspond to the gas-oil ratio of the gas phase, since the production of gas will dominate over the production of oil owing to the mobility of the gas phase being higher than that of the oil phase.

Thus, in this situation, the mobile fluid comprises a highly mobile fluid (the gas phase) and a less mobile fluid (the oil phase), both of which are more mobile than the immobile fluid. In this situation, the composition of the highly mobile fluid is used as the model target, as this is the fluid that will be produced from the reservoir.

The phase behaviour of the mobile fluid can be predicted using a flash algorithm carried out at the respective reservoir conditions in conjunction with the total compositional data obtained from the slim-tube simulations.

From the slim-tube simulations, a plurality of data samples were generated. Simulated reservoir fluid samples were collected from a plurality of spatial locations across each of the simulated reservoirs at a plurality of time locations within the simulation. In the present example, as discussed above, data samples were collected for each of the plurality of spatial locations once at each of the initial state, after having undergone pressure depletion, after having undergone water injection, and after having undergone gas injection.

The simulated reservoir fluid samples include simulated hydrocarbon composition data, which may be in the form of a measurement of the concentration of each hydrocarbon component within the sample, typically covering C₁ to C₃₆₊ hydrocarbons.

FIG. 2 illustrates a workflow 100 for training a machine learning algorithm in order to generate a model for prediction of a gas-oil ratio of free fluid at a location within a reservoir based on measured mud-gas data.

In the following example, an input data set 102 comprising data relating to the simulated reservoir samples is prepared. The input data set 102 comprises target data and input data for each sample and generated from the simulated reservoir fluid samples. The input data corresponds to the data that will be input into the eventual model. The target data corresponds to the desired output of the model.

The input data comprises simulated mud-gas data. Specifically, fully corrected mud-gas data (i.e. where a recycling correction and an extraction efficiency correction have been applied) closely corresponds to the gas composition of the reservoir fluid, e.g. the C₁-C₅ compositions, and consequently the simulated compositions of these fluids may be used as simulated mud-gas data.

The composition data for the mud-gas should comprise data for at least C₁ to C₄ hydrocarbons, and preferably at least C₁ to C₅ hydrocarbons (as is the case in the present example). In some cases, concentrations for up to C₇ or greater hydrocarbons may be included.

The simulated mud-gas data corresponds to the combined compositions of both the mobile fluid and the immobile fluid from the simulated data.

The target data in this example is a gas-oil ratio, and in this example is the single-flash gas-oil measurement of the sample. The gas-oil ratio can be calculated from the compositions of the reservoir properties data, or may be stored as part of the reservoir properties data within the initial data set, i.e. it may be output directly from the reservoir simulations.

The gas-oil ratio is the ratio of the volume of gas that comes out of solution to the volume of oil at surface conditions.

The simulated gas-oil ratio corresponds to the gas-oil ratio of the mobile fluid from the simulated data, or to the gas-oil ratio of the highly mobile fluid in the case of a multi-phase mobile fluid.

In the present embodiment, all of the data points from each of the slim-tube tests was used in the input data set 102. That is to say, the C₁ to C₅ composition of the simulated fluid and the gas-oil ratio of the mobile phase of the simulated fluid at each spatial position along each slim-tube test at every time increment throughout the slim-tube test.

Next, a model generation is performed, in which a model is generated and validated based on the input data set 102.

The input data set 102 is first divided into a training data set 104 and a testing data set 106. The input data set 102 is preferably curated such that at least the testing data set 106 contains data that spans the various classes of the input data set 102 as a whole (e.g. dry gas reservoirs, wet gas reservoirs, oil reservoirs).

Typically, at least 50% of the input data set 102 should be used for training, and at least 10% of the input data set 102 should be used for testing. Common ratios include 50:50, 70:30, 75:25, 80:20, 90:10. However, it will be appreciated that other divisions may be used instead.

Generally the larger the training data set, the more accurate the model will be. However, if too small a test data set is used (or indeed if no test data set is used) then it is not possible to confidently verify the accuracy of the model, e.g. making it difficult to detect an over-fitted model (only accurate for the specific training data).

To generate a model, a machine learning algorithm is provided with the training data set 104, and a set of training parameters to control the machine learning algorithm.

The inventors identified that a Gaussian Process algorithm was the most accurate model, followed by Universal Kriging, Random Forest, KMean and Elastic Net. However, given the noisy nature of the mud gas data, the inventors selected the algorithm based on which produced a model having the greatest stability. The model created using the Random Forest algorithm demonstrated the best performance in terms of providing the greatest stability and was hence used in the demonstration of the method described below.

It will be appreciated that any suitable algorithm may be used. Those operating within this field will be familiar with the procedures for selecting and utilising a machine learning algorithm. Therefore, this will not be discussed in detail.

Model validation 108, e.g. cross-validation, may then then be performed. During the model validation 108, the model is tested to determine how well it predicts new data that was not used in estimating the model, in order to flag problems such as over fitting or selection bias. Model validation 108 is an optional step.

Cross-validation involves partitioning the training data set 104 into complementary subsets, performing the model fitting using one subset of the training data set 104, and validating the analysis on the other subset of the training data set 104. To reduce variability, most methods use multiple rounds of cross-validation, performed using different partitions, and the validation results are combined (e.g. averaged) over the rounds to give an estimate of the model's predictive performance (e.g. a mean average prediction error, MAPE).

In this example K-fold cross-validation, and particularly 4-fold cross-validation is used. In K-fold cross-validation, the training data 104 is separated in K disjoint subsets (in this case, four), known as “folds”. Then, cross-validation is performed by training the model on all of the data except for one fold, and validating the trained model using the fold that was not used for training. The best model is then selected as the model having the best predictive performance, e.g. the lowest MAPE.

A compositional reservoir simulation was carried out to produce a model of a reservoir on which the simulated machine-learning model could be tested to determine its accuracy. The reservoir simulation included the progression of the reservoir from an initial state, through production process involving the injection of gas and water, to a mature state. A machine learning model for predicting a gas-oil ratio of free fluid at a location within the reservoir based on measured mud-gas data was constructed using a training set obtained in relation to this simulated reservoir according to workflow 100 described above. Simulated wells were then drilled at the same location in the simulated reservoir at time points corresponding to the reservoir in an initial state, the reservoir following a period of gas injection, the reservoir following a period of gas and water injection, as well as the reservoir at a time where there is a mobile oil phase and mobile gas phase.

FIGS. 3 to 6 comprise charts comparing the gas-oil ratio of free fluid within the model reservoir, as predicted by two different machine learning models. The first machine learning model is constructed according to the method disclosed in WO 2020/185094 A1. The second machine learning model is constructed according to the method outlined above and described with reference to FIGS. 1 and 2.

The two machine learning models predict the gas-oil ratio of the free fluid at a location within a reservoir based on the mud-gas data. The charts in FIGS. 3 to 6 provide a plot of the gas-oil ratio of the free fluid within the reservoir against depth as predicted by these models.

The first column of each of the plots shows a depth. The second column of each of the plots shows the C₁ to C₅ percentage composition of reservoir fluid at each depth, as would be determined using advanced mud-gas analysis. The third column of each of the plots shows the gas-oil ratio of the free fluid at each depth as predicted by the first machine learning model. The fourth column of each of the plots shows the gas-oil ratio of the free fluid at each depth as predicted by the second machine learning model. The fifth column of each of the plots shows a water saturation percentage, an oil saturation percentage and gas saturation percentage (SWAT, SOIL and SGAS respectively) of the reservoir fluid at each depth.

A true solution to the gas-oil ratio of free fluid vs depth can be determined from the model reservoir produced by the compositional reservoir simulation. The true value of the gas-oil ratio of free fluid within the reservoir when the mobile fluid comprises oil is indicated in the third and fourth columns of each of the plots by a dashed line (RS-oil phase gas oil ratio). The true value of the gas-oil ratio of free fluid within the reservoir when the mobile fluid comprises gas is indicated in the third and fourth columns of each of the plots by the solid black line (1/RV-gas phase gas oil ratio).

The gas-oil ratio of the free fluid within the reservoir as predicted by the first and second machine learning models is plotted in red in their respective columns. The cut-off gas-oil ratio in order to label the reservoir fluids as oil or gas is set to 600 Sm³/Sm³.

FIG. 3 shows the predicted gas-oil ratio of the free fluid within the reservoir when the reservoir is in an initial state. The water saturation percentage, oil saturation percentage and gas saturation percentage profile shows that no gas is present in the reservoir in the initial state. There is good correlation between the true gas-oil ratio of the free fluid within the reservoir from the reservoir model and the gas-oil ratio of the free fluid within the reservoir predicted by both the first and second machine learning models. This is to be expected since both models are constructed using reservoir data based on the initial composition of the reservoir prior to the injection of gas and/or water.

FIG. 4 shows the predicted gas-oil ratio of the free fluid within the reservoir at a time following gas injection and when a gas cap has formed at an upper interval. The upper interval contains maintains a similar gas, oil and water saturation profile to that of the reservoir in the initial condition, as can be seen in the corresponding interval in FIG. 3. Consequently, for the lower interval, the gas-oil ratio of the free fluid within the reservoir predicted by both of the first and second machine learning models match closely with the true gas-oil ratio of the free fluid within the reservoir.

In the upper interval containing the gas cap, the gas-oil ratio of the free fluid within the reservoir predicted by the first machine learning model does not match the true gas-oil ratio of the free fluid within the reservoir. However, the gas-oil ratio of the free fluid within the reservoir predicted by the second machine learning model shows a close match to the true gas-oil ratio of the free fluid within the reservoir even in the upper interval.

FIG. 5 shows the predicted gas-oil ratio of the free fluid within the reservoir at a time following water and gas injection. As in FIG. 4, the gas, oil and water saturation profile of the lower interval of the reservoir remains similar to the initial conditions and there is no significant differences in the gas-oil ratio of the free fluid within the reservoir predicted by the first and second machine learning models. However, the upper interval has now been flooded by the injected water. Since the machine learning models are designed for predicting properties relating to the mobile hydrocarbon phases, the predicted gas-oil ratio of free fluid within the reservoir for these water-filled intervals should be disregarded.

The gas-oil ratio predicted by the second model in the lower interval not flooded by water closely matches the true value.

FIG. 6 shows the predicted gas-oil ratio of free fluid within the reservoir at a time when the reservoir contains a mobile oil phase and a mobile gas phase. The water saturation percentage, oil saturation percentage and gas saturation percentage data indicate the presence of both oil and gas at specific depth intervals. For example, in the interval of 2735 m to 2750 m, both the gas and oil saturation levels are higher than the critical saturation and therefore, there is considered to be both a mobile oil phase and a mobile gas phase. When the overall composition at a specific depth is estimated from the simulated mud-gas data, a built-in flash algorithm calculates the co-existing oil and gas phase compositions. Due to the higher mobility of a gas phase compared to an oil phase, the gas phase gas-oil ratio is estimated as the gas-oil ratio of the free fluid.

Thus, this technique provides significantly improved estimates of reservoir fluid composition within mature reservoirs compared with what was previously possible. This has many potential applications for improving the extraction of oil from such mature reservoirs.

In one example, where a new horizontal production well is being installed at an existing field, mud-gas data is collected as the well bore of the well is drilled. The measured mud-gas data can be examined using the machine-learning model, generated as discussed above to provide a substantially continuous log of the desired property, such as the gas-oil ratio, along a length of the well bore.

Based on this gas-oil ratio log, it can be readily determined which locations of the reservoir along the length of the well bore contain free gas, and which locations contain free oil. Consequently, it is possible to determine one or more locations along the length of the well bore to perforate a casing of the well in order to minimise the risk of producing gas from the well.

Optionally, the gas-oil ratio log may be combined with other data when determining where to perforate the casing. For example, 4D seismic models of the reservoir may be used in combination with this data when determining the perforation locations.

Once the perforation locations have been determine, perforation of the casing is carried out at those locations in a conventional manner that will not be discussed in detail herein.

In a further example, the machine learning model may also be used to examine existing reservoirs. For example, historic mud-gas data collected when wells have been drilled previously may be examined using the machine learning model discussed above to generate a log of the gas-oil ratio along the well at the time it was drilled. This may provide valuable information about the state of the reservoir, as well as potentially identifying additional oil reserves along existing wells that have not previously been produced.

In other examples, the data may not necessarily be used to determine perforation locations, but may simply be displayed to a user on an electronic screen.

1. A method of generating a model for predicting at least one property of a fluid at a sample location within a hydrocarbon reservoir, the method comprising:
simulating behaviour of one or more hydrocarbon reservoirs during production;
generating a plurality of simulated fluid samples from the one or more simulated hydrocarbon reservoirs, the plurality of simulated fluid samples corresponding to a plurality of different spatial locations and/or different time locations within the one or more simulated hydrocarbon reservoirs;
generating a training data set comprising input data and target data based on the simulated fluid samples, the input data comprising simulated mud-gas data for each sample location indicative of mobile and immobile hydrocarbons at the sample location, and the target data comprising the at least one property of only the mobile hydrocarbons at each sample locations; and
constructing a model using the training data set such that the model can be used to predict the at least one property of the fluid at a sample location based on measured mud-gas data for the sample location.

2. The method according to claim 1, wherein the plurality of simulated fluid samples correspond to time locations comprising one or more of:
a time when the simulated reservoir is at an initial state;
a time when the simulated reservoir is undergoing or has undergone pressure depletion;
a time when the reservoir is undergoing or has undergone water injection; and
a time when the reservoir is undergoing or has undergone gas injection.

3. The method according to claim 1, wherein the at least one property comprises a gas-oil ratio of the mobile fluid at the sample location.

4. The method according to claim 1, wherein the at least one property comprises a density of the mobile fluid at the sample location.

5. The method according to claim 1, wherein the at least one property comprises a saturation pressure of the mobile fluid at the sample location.

6. The method according to claim 1, wherein the at least one property comprises a formation volume factor of the mobile fluid at the sample location.

7. The method according to claim 1, wherein the at least one property comprises a concentration of a C₇₊ hydrocarbon within the mobile fluid at the sample location.

8. The method according to claim 1, wherein the mud-gas data is indicative of a concentration of C₁ to C₅ hydrocarbon gases at the sample location.

9. The method according to claim 1, wherein simulating the behaviour of one or more hydrocarbon reservoir during production is performed using slim-tube simulations.

10. A computer-based model for predicting at least one property of a fluid at a sample location within a hydrocarbon reservoir based on measured mud-gas data for that sample location, the computer-based model having been generated by the method according to claim 1.

11. A tangible computer-readable medium storing the computer-based model of claim 10.

12. A method of predicting a value of a fluid property of a fluid at a sample location within a hydrocarbon reservoir, the method comprising:
providing measured mud-gas data for the sample location; and
predicting the values of a fluid property of the fluid at the sample location by supplying the measured mud-gas data to the model according to claim 10.

13. A method of predicting a value of a fluid property of a fluid along a length of a well through a hydrocarbon reservoir, the method comprising:
predicting a value of a fluid property of a fluid at a plurality of sample locations along a length of a well using the method according to claim 10.

14. The method according to claim 13, further comprising:
displaying, using an electronic display screen, a graph plotting the predicted value of the fluid property against a location of the respective sample location for each of the plurality of sample locations along the length of the well.

15. A method comprising:
drilling a well bore through a hydrocarbon reservoir, wherein mud-gas data is collected as the well is drilled;
predicting a value of a fluid property of a fluid at a plurality of sample locations along a length of the well bore using the method according to claim 10;
determining at least one perforation location along the well bore, based on the predicted value of the fluid property of the fluid; and
perforating a casing of the well bore at the determined at least one perforation location.

16. The method according to claim 15, wherein the well bore is a horizontal well bore.

17. The method according to claim 15, wherein the well bore is a production well bore.

18. The method according to claim 15, wherein the reservoir is a mature reservoir.
